(19) United States
(12) Patent Application Publication
(10) Pub. No.: US 2009/0274722 A1
(43) Pub. Date: Nov. 5, 2009

(54) Therapeutic Composition for the Treatment of BPH and ED

(76) Inventor: John E. Ligums, Houston, TX (US)

Correspondence Address: SUMMA, ADDITON & ASHE, P.A., 11610 NORTH COMMUNITY HOUSE ROAD, SUITE 200, CHARLOTTE, NC, 28277, US

(21) Appl. No.: 12/503,449

(22) Filed: Jul. 15, 2009

Related U.S. Application Data
(63) Continuation-in-part of application No. PCT/US2008/052244, filed on Jan. 29, 2008.
(60) Provisional application No. 60/887,036, filed on Jan. 29, 2007.
(60) Provisional application No. 61/085,142, filed on Jul. 31, 2008.

(51) Int. Cl. A61K 36/06; A61K 36/906

(52) U.S. Cl. 424/195.15; 424/756

(57) ABSTRACT

The invention relates to a therapeutic composition for the prevention and/or treatment of Benign Prostatic Hyperplasia (BPH) and other related prostate problems. The invention is a therapeutic composition that combines alfalfa and fennel to have a synergistic effect for the treatment of prostate problems, erectile dysfunction, and/or sleep disorders. The composition may further include ginger, red Reishi mushroom and oyster mushroom.

### CROSS-REFERENCE TO PRIORITY APPLICATIONS

This application is a continuation-in-part of International Patent Application No. PCT/US2008/052244 for Therapeutic BPH Composition, filed Jan. 29, 2008, (and published Aug. 7, 2008, as Publication No. WO 2008/094873), which itself claims the benefit of U.S. Provisional Patent Application No. 60/887,036, for Therapeutic BPH Composition, filed Jan. 29, 2007.

The present application further claims the benefit of U.S. Provisional Patent Application Ser. No. 61/085,142, for Therapeutic Composition for the Treatment of BPH and ED, filed Jul. 31, 2008.

Each of the foregoing patent application publications and patent applications is hereby incorporated by reference in its entirety.

### FIELD OF THE INVENTION

The invention relates to a therapeutic composition for the treatment (e.g., the prevention and/or cure) of prostate problems (such as Benign Prostatic Hyperplasia (BPH)) and for the improvement of sexual performance in men (e.g., by alleviating problems associated with erectile dysfunction).

### BACKGROUND OF THE INVENTION

The prostate is a gland within the male reproductive system located just below the bladder. After puberty, the prostate gradually grows from roughly the size of a grape to the size of a golf ball. At around age 50, the prostate resumes growing. This increased growth can cause various problems, most commonly Benign Prostatic Hypertrophy (BPH), prostatitis, and even prostate cancer.

The specific cause of these problems is generally unknown, but is thought to be related to hormonal changes brought on by aging. As men age, an increased amount of hormones (e.g., androgen, estrogen and prolactin) decrease the rate at which testosterone and the dihydrotestosterone (DHT) are eliminated from the prostate. (Those having ordinary skill in the art will recognize that testosterone is converted into DHT by 5-alpha-reductase.) This excessive accumulation of DHT is thought to be responsible for prostate problems.

Benign prostatic hypertrophy (BPH) (i.e., enlarged prostate) occurs when, in response to hormones such as testosterone, the cells in the inner core of the prostate gland grow and form fibrous nodules. These nodules put pressure on the urethra causing pain and problems urinating. If left untreated, BPH can lead to prostatitis.

Common symptoms of BPH include a need to urinate frequently, pain, burning, blood in the urine, weak urine flow, nighttime wakening to empty the bladder, and generally a constant feeling that the bladder is full.

Drugs are often used to treat moderate cases of BPH. Such drugs include alpha-blockers, which make it easier to urinate by relaxing the muscles located where the bladder narrows toward the urethra. Other such drugs include finasteride or dutasteride, each of which shrinks the prostate gland. Finasteride and dutasteride, however, may achieve noticeable improvements only for men with significantly enlarged prostates. It is generally thought that a combination of these two kinds of drugs (i.e., alpha-blockers and finasteride or dutasteride) is more beneficial than either drug alone.

There are concerns about using conventional drugs. Some drugs are habit forming and even addictive; others have harmful side effects. Thus, alternative medicines are becoming increasingly popular. Alternative medicines are not only safe (e.g., do not have side effects) but also frequently work better than conventional medicines for the prevention and treatment of chronic disease. Moreover, alternative medicines may better satisfy the mental, emotional, and spiritual needs of an individual.

It is well known that some herbs work better when used in combination rather than when used alone. Combining herbs with similar but different properties increases efficacy such that the total effect from the combination of herbs is greater than the sum of the individual effects provided by the respective herbs. In other words, the combination of herbs can provide a synergistic effect for the patient.

Several patents disclose herbal remedies for preventing and treating problems related to prostate.

U.S. Pat. No. 7,022,350 discloses the use of palmetto as a dietary supplement for treating prostate disease.

U.S. Pat. No. 6,261,607 discloses an herbal composition prepared from saw palmetto, green tea, pumpkin seed oil, ginger, dual urtica root extracts, selenium, watermelon, and rosemary to promote prostate health.

U.S. Pat. No. 6,790,464 discloses compositions prepared from herbs (e.g., radix asparagi, radix angelicae, and pubescentis) for the prevention or treatment of prostate conditions.

U.S. Pat. No. 6,482,447 discloses compositions comprising lycopene, serenoa repens, pygeum africanum, and Urtica dioica for the treatment of Benign Prostate Hypertrophy (BPH) and the prevention of prostate cancer.

It is recognized that different patients respond differently to different treatments. Therefore, there is a need for additional herbal compositions and methods for preventing and treating problems related to the prostate gland.

Drugs are often used to treat erectile dysfunction (ED). These drugs all work by increasing blood flow into the penis, thereby allowing for an erection suitable for sexual intercourse.

As indicated above, it is desirable to have an alternative herbal medicine to the oral drugs currently used to treat erectile dysfunction (ED).

### SUMMARY OF THE INVENTION

In one aspect, the invention is a composition for the relief and treatment of prostate problems (e.g., BPH).

In another aspect, the invention is a composition for the prevention of prostate disorders (e.g., BPH).

In yet another aspect, the invention is a composition for the cure of prostate disorders (e.g., BPH).

In yet another aspect, the invention is a composition for the improvement of male sexual performance (e.g., alleviation of erectile dysfunction).

In yet another aspect, the invention is a composition for improving sleep and alleviating sleep disorders.

In yet another aspect, the invention is a composition that includes a synergistic combination of phytoestrogenic compounds (e.g., isoflavones, coumestans, and lignans).

In yet another aspect, the invention is a composition that includes a synergistic combination of alfalfa and fennel.

In yet another aspect, the invention is a composition that includes a combination of alfalfa, fennel, and ginger.

In yet another aspect, the invention is a composition that includes a synergistic combination of alfalfa and fennel, and optionally red Reishi mushroom and/or oyster mushroom.

In yet another aspect, the invention is a composition that includes a synergistic combination of alfalfa, fennel, and ginger, and optionally red Reishi mushroom and/or oyster mushroom.

In yet another aspect, the invention is a process for the preparation of various therapeutic compositions for the relief and treatment of prostate problems.

In yet another aspect, the invention is a therapeutic method for the relief, treatment, prevention, and/or cure of prostate problems.

In yet another aspect, the invention is a process for the preparation of various therapeutic compositions for the achievement of improved sexual performance in men.

In yet another aspect, the invention is a therapeutic method for the achievement of improved sexual performance in men.

In yet another aspect, the invention is a therapeutic method for improving sleep and alleviating sleep disorders (e.g., fatigue, sleep deprivation, and/or insomnia).

The foregoing, as well as other objectives and advantages of the invention and the manner in which the same are accomplished, are further specified within the following detailed description.

### DETAILED DESCRIPTION

The invention is a therapeutic composition for the prevention and/or treatment of prostate problems (e.g., BPH, and prostatitis) and for the improvement of sexual performance in men. The invention is also a therapeutic composition for alleviating sleep disorders.

In one embodiment, the invention is a therapeutic composition formed from alfalfa, fennel, and ginger.

Alfalfa (scientifically known as Medicago Sativa) is a member of the legume family. An herbaceous perennial, it grows in various climates throughout the world. Alfalfa is rich in minerals and nutrients, including calcium, magnesium, potassium, carotene, and vitamins K and E. In addition, alfalfa leaves contain eight essential amino acids. Alfalfa is recognized as being useful, for example, for the treatment of urinary tract infections, heart disease, and even cancer.

Alfalfa contains phytoestrogens (e.g., Biochanin-A, Coumestrol, Apigenin, Luteolin, Quercetin, Formononetin, Genistein, and Daidzein). Phytoestrogens are compounds that possess oestrogenic properties. Most phytoestrogens belong to a large group of substituted phenolic compounds known as flavonoids.

Phytoestrogens (i.e., flavonoids) in alfalfa help to balance testosterone levels (e.g., decrease excessive amounts of testosterones), thus inhibiting the enlargement of prostate. In this way, it is thought that these phytoestrogens in alfalfa help to prevent and/or treat BPH and related prostate problems.

Fennel (scientifically known as Foeniculum vulgare) is a highly aromatic perennial herb that grows to six feet or so. Fennel, which has a flavor similar to that of anise and star anise, has been used as herbal medication in the past (e.g., to treat chills, stomach disorders and kidney stones). It also has culinary uses in several parts of the world including India, Italy, and the Middle East.

Like alfalfa, fennel contains phytoestrogenic compounds (e.g., anethole, kaempherol, beta-sitosterol, eugenol, ferulic acid, quercetin, and rutin) that help balance testosterone levels. It is thus believed that the phytoestrogens in fennel help to treat BPH and other related prostate problems.

Those having ordinary skill in the art will appreciate that blending certain herbs can enhance the therapeutic benefits of a particular drug or medicinal supplement. Similarly, combining alfalfa and fennel together has a synergistic effect that helps broaden its therapeutic range (e.g., the isoflavone coumestrol in alfalfa and the isoflavone anethole in fennel have a synergistic effect in treating prostate problems).

Ginger (Zingiber officinale), which may also be used as an additional ingredient in the present therapeutic composition, is perennial plant with an edible underground stem (i.e., a rhizome). Its rhizome extends roughly 12 inches above ground with long, narrow, ribbed, green leaves, and white or yellowish-green flowers.

Those skilled in the field of herbal medicine know that ginger has been used as a medicine in Asia and Arabia since ancient times. In China, for example, ginger has been used for more than 2,000 years to aid digestion and treat stomach upset, diarrhea, and nausea. Ginger has also been used to help treat arthritis, colic, diarrhea, and heart conditions. In addition to medicinal uses, ginger is also used as an important cooking spice and is believed to help alleviate the symptoms of the flu and common cold.

The pungency of ginger is caused by a non-volatile resin containing the same kind of hydroxyaryl compound found in other spices of the ginger family (i.e., zingerone, gingeroles, and shogoales). During storage, the pungent gingeroles degrade to the milder shogoales. Gingeroles and shogoales are the active constituents in ginger that reduce inflammation. Accordingly, it is thought that, with respect to the present invention, ginger provides relief from the inflammation associated with prostate problems. It is further thought that, with respect to the present invention, the anti-inflammatory properties of ginger may promote blood vessel health. This, in turn, may allow for greater blood flow to the penis and thereby improve sexual performance in men.

In accordance with the foregoing, the therapeutic composition of the present invention may include alfalfa plant extracts (e.g., dried or aqueous), alfalfa leaves, alfalfa seeds, and/or alfalfa flowers. It may further include fennel plant extracts, fennel leaves, and/or fennel seeds. Additionally, it may include ginger extracts and/or ginger rhizome.

In another embodiment, the invention is a therapeutic composition formed from alfalfa, fennel, ginger, red Reishi mushroom, and/or oyster mushroom.

Red Reishi mushroom (scientifically known as Ganoderma lucidum) has been used as an herbal medicine in Japan and China for over 2,000 years. It is mainly composed of complex carbohydrates (i.e., water-soluble polysaccharides), triterpenes, proteins, and amino acids. The water-soluble polysaccharides found in red Reishi mushroom seem to be useful in lowering blood pressure and preventing and/or treating tumors. Another active ingredient in red Reishi mushroom is triterpenes (e.g., ganoderic acids). Preliminary studies have indicated that ganoderic acids help alleviate common allergies by inhibiting histamine release, improving oxygen utilization, and improving liver functions. Accordingly, it is thought that, with respect to the present invention, red Reishi mushroom helps in preventing and/or treating the tumor associated with prostate related problems. It is further thought that, with respect to the present invention, the blood-pressure-reducing qualities of red Reishi mushroom may improve blood flow to the penis, thereby improving sexual performance.

Oyster mushroom (scientifically known as Pleurotus ostreatus) is an edible fungus that may reduce cholesterol levels and have anti-cancer properties, too. Traditionally, it has been used to strengthen veins and relax tendons. Dried oyster mushroom, which is high in iron, is helpful to maintain a healthy blood system (e.g., increase red blood cells and perhaps improve blood flow to the prostate and/or penis).

In addition, oyster mushroom also contains carbohydrates, glucan, polysaccharo-peptides, triterpenes, fiber, vitamins B1, B2, minerals, and antioxidants. It seems that the polysaccharides of oyster mushroom are ingested by macrophages (i.e., immune cells) and are transported to other immune cells. This leads to the increase in number of natural killer cells that are responsible for preventing and/or inhibiting tumors. Accordingly, with respect to the present invention, it is thought that oyster mushroom assists in preventing and/or treating tumors associated with the prostate.

In yet another embodiment, the invention is a composition formed from phytoestrogenic compounds (e.g., Genestein and Gegnis) that may be found in alfalfa and fennel. These compounds have a synergistic therapeutic effect for preventing and/or treating BPH and enhancing sexual performance in men.

In yet another embodiment, the invention is a composition formed from the combination of phytoestrogens, such as those found in alfalfa and fennel. Further, the invention is a composition that may be formed from gingeroles and shogoales such as found in ginger. Furthermore, the composition may include ingredients such as those found in red Reishi mushroom and/or oyster mushroom (e.g., polysaccharides, triterpenes, proteins, and amino acids).

The therapeutic composition of the present invention typically includes between about 20 and 60 weight percent of alfalfa. Typically, the composition includes between about 25 and 45 weight percent of alfalfa. More typically, the composition includes about 40 weight percent of alfalfa (e.g., between about 35 and 45 weight percent).

The therapeutic composition of the present invention further includes between about 5 and 35 weight percent of fennel. Typically, the composition includes between about 10 and 25 weight percent of fennel. More typically, the composition includes about 15 weight percent of fennel (e.g., between about 10 and 20 weight percent).

The therapeutic composition of the present invention may further include between about 1 and 15 weight percent of ginger. Typically, the composition may include between about 1 and 10 weight percent of ginger. More typically, the composition may include about 5 weight percent of ginger (e.g., between about 2 and 8 weight percent).

Further, the therapeutic composition may include between about 10 and 35 weight percent of red Reishi mushroom. Typically, the composition includes between about 15 and 30 weight percent of red Reishi mushroom. More typically, the composition includes about 20 weight percent of red Reishi mushroom (e.g., between about 15 and 25 weight percent).

Furthermore, the therapeutic composition may include between about 10 and 35 weight percent of oyster mushroom. Typically, the composition includes between about 15 and 30 weight percent of oyster mushroom. More typically, the composition includes about 20 weight percent of oyster mushroom (e.g., between about 15 and 25 weight percent).

In yet another aspect, the invention is a process for the preparation of a therapeutic composition for the relief and treatment of prostate problems.

In one aspect, the composition may be prepared by mixing the plant extracts of alfalfa and fennel.

In another aspect, the composition may be prepared by mixing the plant extracts of alfalfa, fennel, and ginger.

In yet another aspect, the composition may be prepared by mixing the plant extracts of alfalfa, fennel, ginger, and the extracts of red Reishi mushroom and/or oyster mushroom.

In yet another aspect, the invention is a method for providing the therapeutic composition for prevention and/or treatment of prostate problems. In this regard, the composition may be administered orally to an adult male in the form of a capsule, tablet, powder, or liquid solution in an amount of at least about 250 milligrams per day (e.g., in an amount of between about 500 milligrams and 1.5 grams per day). More typically, the composition may be administered in twice-daily dosages of 250 milligrams.

A preliminary clinical trial followed twelve human male subjects taking an oral dosage of a therapeutic composition according to the present invention. For this clinical trial, a composition including about 40 weight percent alfalfa, about 15 weight percent fennel, about 5 weight percent ginger, about 20 weight percent red Reishi mushroom extract, and about 20 percent oyster mushroom extract was administered to twelve human male subjects twice daily in the amount of 250 milligrams per dose for fourteen days.

As evidence of the invention's efficacy vis-à-vis relief of enlarged prostate symptoms, all twelve subjects reported improvement of prostate-related symptoms at the end of the trial. In this regard, the twelve test subjects maintained diaries over the duration of the clinical trial. Each test subject recorded (i.e., self-reported on a ten-point scale questionnaire) his prostate-related symptoms (i.e., the urge to urinate, the ability to initiate urination, and strength of urine stream).

With respect to the trial study, all twelve study participants reported a decrease in the urgency to urinate. For each study participant, this decrease ranged from about 70 to 90 percent below self-reported baseline, with an average decrease of over 80 percent from an average baseline of 8.2.

With respect to the trial study, all twelve participants reported an increase in the ability to initiate urination. For each study participant, this increase ranged from about 65 to 85 percent above self-reported baseline, with an average increase of 75 percent from an average baseline of 2.5.

With respect to the trial study, all twelve participants reported an increase in the force of the urine stream. For each study participant, this increase ranged from 65 to 85 percent above self-reported baseline, with an average increase of more than 75 percent from an average baseline of 2.4.

As an aside, three of the study participants reported sleep improvement (and thus relief from fatigue caused by sleep deprivation). Without being bound to any theory, it is thought that the decreased urge to urinate may partly explain the alleviation of sleep disorders.

As evidence of the invention's efficacy vis-à-vis improved sexual performance in men, four of the twelve subjects in the study reported an increase in the ability to achieve and maintain an erection. Furthermore, these four subjects also reported a general increase in libido by the end of the study.

In yet another aspect, the invention is a method for providing the therapeutic composition for improvement of male sexual performance. In this regard and by way of example, the composition as disclosed herein may be administered orally to an adult male in the form of a capsule, tablet, powder, or liquid solution in an amount of at least about 250 milligrams per day (e.g., in an amount of between about 500 milligrams and 1.5 grams per day). More typically, the composition may be administered in twice-daily dosages of 250 milligrams.

Indeed, preliminary trials suggest that an unexpected and beneficial side effect is that the compositions according to the present invention may improve sexual performance in men. Specifically, three participants in the preliminary clinical study reported an increase in the ability to achieve an erection and a general increase in libido.

This indication of improved sexual performance may prove beneficial for men who suffer from sexual disorders, such as erectile dysfunction, commonly referred to as ED. Erectile dysfunction is the inability to sustain an erection suitable for sexual intercourse. Because an erection is caused by the flow of blood into the penis, conditions that affect blood flow (e.g., heart disease) are a common cause of erectile dysfunction.

In the specification, typical embodiments of the invention have been disclosed. The present invention is not limited to such exemplary embodiments. Unless otherwise noted, specific terms have been used in a generic and descriptive sense and not for purposes of limitation.

1. A therapeutic composition, comprising:
alfalfa;
fennel; and
ginger.

2. A therapeutic composition according to claim 1, wherein said alfalfa comprises alfalfa extracts, alfalfa leaves, alfalfa seeds, and/or alfalfa flowers.

3. A therapeutic composition according to claim 1, wherein said fennel comprises fennel extracts, fennel leaves, and/or fennel seeds.

4. A therapeutic composition according to claim 1, wherein said ginger comprises ginger extracts and/or ginger rhizome.

5. A therapeutic composition according to claim 1, consisting essentially of:
alfalfa;
fennel; and
ginger.

6. A therapeutic composition according to claim 1, wherein said alfalfa is present in said therapeutic composition in an amount between about 20 and 60 weight percent.

7. A therapeutic composition according to claim 1, wherein said alfalfa is present in said therapeutic composition in an amount between about 30 and 50 weight percent.

8. A therapeutic composition according to claim 1, wherein said fennel is present in said therapeutic composition in an amount between about 5 and 35 weight percent.

9. A therapeutic composition according to claim 1, wherein said fennel is present in said therapeutic composition in an amount between about 10 and 25 weight percent.

10. A therapeutic composition according to claim 1, wherein said ginger is present in said therapeutic composition in an amount between about 1 and 15 weight percent.

11. A therapeutic composition according to claim 1, wherein said ginger is present in said therapeutic composition in an amount between about 1 and 10 weight percent.

12. A therapeutic composition according to claim 1, wherein:
said alfalfa is present in an amount between about 30 and 65 weight percent;
said fennel is present in an amount between about 5 and 40 weight percent; and
said ginger is present in an amount between about 1 and 20 weight percent.

13. A therapeutic composition according to claim 1, further comprising red Reishi mushroom.

14. A therapeutic composition according to claim 13, consisting essentially of:
alfalfa;
fennel;
ginger; and
red Reishi mushroom.

15. A therapeutic composition according to claim 13, wherein said red Reishi mushroom is present in said therapeutic composition in an amount between about 5 and 35 weight percent.

16. A therapeutic composition according to claim 13, wherein said red Reishi mushroom is present in said therapeutic composition in an amount between about 10 and 30 weight percent.

17. A therapeutic composition according to claim 1, further comprising oyster mushroom.

18. A therapeutic composition according to claim 17, consisting essentially of:
alfalfa;
fennel;
ginger; and
oyster mushroom.

19. A therapeutic composition according to claim 17, wherein said oyster mushroom is present in said therapeutic composition in an amount between about 5 to 35 weight percent.

20. A therapeutic composition according to claim 17, wherein said oyster mushroom is present in said therapeutic composition in an amount between about 10 to 30 weight percent.

21. A therapeutic composition according to claim 1, further comprising;
red Reishi mushroom; and
oyster mushroom.

22. A therapeutic composition according to claim 21, consisting essentially of:
alfalfa;
fennel;
ginger;
red Reishi mushroom; and
oyster mushroom.

23. A therapeutic composition according to claim 21, wherein said red Reishi mushroom is present in said therapeutic composition in an amount between about 5 and 40 weight percent.

24. A therapeutic composition according to claim 21, wherein said oyster mushroom is present in said therapeutic composition in an amount between about 5 and 40 weight percent.

25. A therapeutic composition according to claim 21, wherein:
said alfalfa is present in an amount between about 30 and 60 weight percent;
said fennel is present in an amount between about 5 and 35 weight percent;
said ginger is present in an amount between about 1 and 15 weight percent;
said red Reishi mushroom is present in an amount between about 10 and 30 weight percent; and
said oyster mushroom is present in an amount between about 10 and 30 weight percent.

26. A therapeutic composition according to claim 21, wherein:
said alfalfa is present in an amount between about 25 and 55 weight percent;
said fennel is present in an amount between about 5 and 20 weight percent;
said ginger is present in an amount between about 2 and 8 weight percent;
said red Reishi mushroom is present in an amount between about 10 and 25 weight percent; and
said oyster mushroom is present in an amount between about 10 and 25 weight percent.

27. A method of providing the therapeutic composition of claim 1, comprising administering said composition to an adult male.

28. A method of providing the therapeutic composition according to claim 27, comprising administering said composition to an adult male with prostate problems.

29. A method of providing the therapeutic composition according to claim 27, comprising administering said composition to an adult male with BPH.

30. A method of providing the therapeutic composition according to claim 27, comprising administering said composition to an adult male with decreased libido.

31. A method of providing the therapeutic composition according to claim 27, comprising administering said composition to an adult male with erectile dysfunction.

32. A method of providing the therapeutic composition according to claim 27, comprising administering said composition to alleviate one or more sleep disorders.

33. A method of providing the therapeutic composition according to claim 27, wherein said therapeutic composition is orally administered in capsule form.

34. A method of providing the therapeutic composition according to claim 27, wherein said therapeutic composition is orally administered in tablet form.

35. A method of providing the therapeutic composition according to claim 27, wherein said therapeutic composition is orally administered in powder form.

36. A method of providing the therapeutic composition according to claim 27, wherein said therapeutic composition is orally administered in liquid solution form.

37. A method of providing the therapeutic composition according to claim 27, wherein said therapeutic composition is administered up to five times per day.

38. A method of providing the therapeutic composition according to claim 27, wherein said therapeutic composition is administered in an amount between about 1 to 4 grams per day.
